(19) United States
(12) Patent Application Publication
(10) Pub. No.: US 2026/0334755 A1
(43) Pub. Date: Oct. 29, 2026

(54) MEDICAL DEVICES, SYSTEMS, AND METHODS FOR EVALUATING VISUALIZATION

(71) Applicant: Boston Scientific Scimed, Inc., Maple Grove, MN (US)

(72) Inventors: Jessica WILLIAMS, Marlborough, MA (US); Longquan CHEN, Lexington, MA (US); Candace A. RHODES, Hopkinton, MA (US); Alycia ABBOTT, Groton, MA (US); Anya S. CHASE, Dover, NH (US); Niraj Prasad RAUNIYAR, Plymouth, MN (US)

(73) Assignee: Boston Scientific Scimed, Inc., Maple Grove, MN (US)

(21) Appl. No.: 19/655,124

(22) Filed: Apr. 22, 2026

Related U.S. Application Data
(60) Provisional application No. 63/794,568, filed on Apr. 25, 2025.

(51) Int. Cl. A61B 1/00; A61B 1/015

(52) U.S. Cl. CPC A61B 1/00009; A61B 1/00006; A61B 1/0002; A61B 1/0004; A61B 1/00045; A61B 1/00055; A61B 1/00066; A61B 1/00128; A61B 1/015

(57) ABSTRACT

Medical devices, systems, and methods for evaluating visualization are described. The medical system may include a handle, a shaft, and a processor. The handle may include a port configured to be coupled to a fluid management system. The shaft may extend distally from the handle to a distal end. The shaft may define a fluid channel in communication with the port. A distal portion of the shaft may include an imaging device. The processor may be communicatively connected to the imaging device. The processor may be configured to receive one or more image frames from the imaging device, analyze pixels for each frame of the one or more image frames, calculate a visualization score for the one or more image frames based on the analyzed pixels of the respective image, and relay instructions to the fluid management system to adjust a fluid flow based on the visualization score.

### CROSS-REFERENCE TO RELATED APPLICATIONS

This application claims the benefit of priority to U.S. Provisional Application No. 63/794,568, filed on Apr. 25, 2025, which is incorporated by reference herein in its entirety.

### TECHNICAL FIELD

Various aspects of this disclosure generally relate to medical systems, medical devices and related methods associated with visualization of target sites in medical procedures. In particular, some aspects of this disclosure relate to actions to adjust visualization features using metrics of the medical systems.

### BACKGROUND

During medical procedures, such as urological and endoscopic procedures, a medical professional may insert a medical device into a patient, and guide that medical device through tortuous anatomy for positioning the device at a target treatment site in the body. For example, the medical professional may insert a ureteroscope into urinary passages to diagnose and/or treat tissues of the urinary tract. During a procedure, materials such as irrigation fluids, bodily fluids, and tissues and other particulates often obscure a visualization field of the medical device. For example, laser lithotripsy may generate particulates when laser energy is applied to a kidney stone to break it apart for removal. An obscured visualization field may require actions including pausing a procedure, sometimes multiple times, until the visualization field is sufficiently cleared to safely resume. Accordingly, an obscured visualization field may increase procedure times and potentially increase risks for patients.

### SUMMARY

Each of the aspects disclosed herein may include one or more features described in connection with any of the other disclosed aspects.

Aspects of this disclosure include medical devices, systems, and related methods for evaluating visualization. For example, this disclosure includes a medical system comprising a handle, a shaft, and a processor. The handle may include a port configured to be coupled to a fluid management system. The shaft may extend distally from the handle to a distal end of the shaft. The shaft may define a fluid channel in communication with the port. A distal portion of the shaft may include an imaging device. The processor may be communicatively connected to the imaging device. In some examples, the processor may be configured to receive one or more image frames from the imaging device, analyze pixels for each frame of the one or more image frames, calculate a visualization score for the one or more image frames based on the analyzed pixels of the respective image, and relay instructions to the fluid management system to adjust a fluid flow through the fluid channel based on the visualization score.

The one or more image frames may include a plurality of image frames, e.g., wherein the processor is configured to calculate an aggregate score of a plurality of visualization scores of the plurality of image frames. The aggregate score may be, for example, a mean, median, or standard deviation of the plurality of visualization scores. Optionally, the medical system may further include a display in communication with the processor. The display may include a user interface, e.g., including a parameter indicator associated with a parameter of the fluid flow or a visualization score of at least one image frame of the plurality of image frames. The parameter indicator may include a graphic representation. In some examples, the shaft further defines a working channel that may receive a laser device. The processor may be configured to relay instructions to the laser device to adjust laser energy delivered at the distal end of the shaft based on the visualization sore. Additionally or alternatively, the processor may be configured to relay instructions to the fluid management system to adjust a pressure of the fluid flow through the fluid channel.

In some examples, the processor may be configured to compare the visualization score to a predetermined threshold. For example, the processor may be configured to instruct the fluid management system to increase fluid flow through the fluid channel if the visualization score is less than or equal to the predetermined threshold. Additionally or alternatively, the processor may be configured to instruct the fluid management system to maintain or decrease fluid flow through the fluid channel if the visualization score is greater than the predetermined threshold. Further, in some examples, the processor may be configured to generate an alert if the visualization score is less than or equal to the predetermined threshold. The predetermined threshold may be a standard deviation of one or more visualization scores from a previous medical procedure, for example.

In some examples according to this disclosure, the processor may be configured to calculate an amount of time elapsed during which the visualization score is less than a predetermined threshold. The processor may be configured to instruct the fluid management system to increase fluid flow through the fluid channel when the time elapsed exceeds the predetermined threshold.

This disclosure also includes a medical system comprising a handle, a shaft, a display, and a processor. The handle may include a port configured to be coupled to a fluid management system. The shaft may extend distally from the handle to a distal end of the shaft. The shaft may define a fluid channel in communication with the port. A distal portion of the shaft may include an imaging device. The processor may be communicatively connected to the imaging device and the display. The processor may be configured to receive a plurality of image frames from the imaging device, analyze pixels for each frame of the plurality of image frames, calculate a visualization score for each image frame based on the analyzed pixels of the respective image, and relay instructions to the fluid management system to adjust a fluid flow through the fluid channel based on the visualization scores.

The processor may include a memory that stores a threshold visualization score. For example, the processor may be configured to calculate an amount of time elapsed during which the visualization score is less than a predetermined threshold (the threshold visualization score, for example). The processor may be configured to instruct the fluid management system to increase fluid flow through the fluid channel when the time elapsed exceeds the predetermined threshold. The display may include a user interface. The user interface may include a parameter indicator configured to graphically display a visualization score of at least one image frame of the plurality of image frames or a parameter of the fluid flow.

This disclosure also includes a medical system that comprises a handle, a shaft, a display, and a processor. The handle may include a port configured to be coupled to a fluid management system. The shaft may extend distally from the handle to a distal end of the shaft. The shaft may define a fluid channel in communication with the port. A distal portion of the shaft may include an imaging device. The processor may include a memory and may be communicatively connected to the imaging device and the display. The processor may be configured to receive a plurality of image frames from the imaging device, analyze pixels for each frame of the plurality of image frames, calculate a visualization score for each image frame based on the analyzed pixels of the respective image, compare the visualization score to a predetermined threshold stored by the memory, and relay instructions to the fluid management system to adjust a fluid flow through the fluid channel based on the visualization scores.

### BRIEF DESCRIPTION OF FIGURES

The accompanying drawings, which are incorporated in and constitute a part of this application, illustrate exemplary aspects that, together with the written descriptions, serve to explain the principles of this disclosure. Each figure depicts one or more exemplary aspects according to this disclosure, as follows:

FIG. 1 depicts an exemplary medical system according to some aspects of the disclosure.

FIGS. 2A-2D depict various views of an exemplary user interface of the medical system at different points in time during an exemplary medical procedure.

FIG. 3 depicts a flow chart of an exemplary method according to aspects of the disclosure.

### DETAILED DESCRIPTION

The following description sets forth exemplary aspects of the disclosure. It should be recognized, however, that such description is not intended as a limitation on the scope of the disclosure. Rather, the description also encompasses combinations and modifications to those exemplary aspects described herein.

It may be understood that both the foregoing general description and the following detailed description are exemplary and explanatory only and are not restrictive of the invention, as claimed. As used herein, the terms “comprises,” “comprising,” “has,” “having,” “includes,” “including,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “diameter” may refer to a width where an element is not circular. The term “distal” refers to a direction away from an operator, and the term “proximal” refers to a direction toward an operator. The term “exemplary” is used in the sense of “example,” rather than “ideal.” The term “approximately,” or like terms (e.g., “substantially” or “about”), includes values +/−10% of a stated value. Unless otherwise stated, ranges disclosed herein include the end points of the ranges. The term “or” is used disjunctively, such that “at least one of A or B” includes, (A), (B), (A and A), (A and B), etc. For example, the term “or” also includes “and/or.” The term “distal” refers to a portion farthest away from a user when introducing a device into a patient. By contrast, the term “proximal” refers to a portion closest to the user when placing the device into the patient.

Although ureteroscopes are referenced herein, reference to ureteroscopes should not be construed as limiting the medical systems and devices and/or possible applications of the disclosed aspects. For example, the disclosed aspects may be used with endoscopes, duodenoscopes, bronchoscopes, colonoscopes, catheters, sheaths, diagnostic or therapeutic tools or devices, or other types of medical devices.

Aspects of various devices are now described. Some aspects are described with reference to noninvasive procedures, such as urology procedures. For example, the distal end of a medical device may include a light source and imaging device, e.g., for viewing the target site. The medical device also may include a working channel to advance an instrument such as a laser fiber for delivering laser energy to the target site, e.g., to fragment a stone such as a kidney stone. Another exemplary instrument that may be advanced through the working channel may include an end effector, such as a snare or forceps, to capture and extract a stone or a stone fragment. References to a particular type of medical procedure, such as a urological procedure; organ, such as a kidney; and/or object, such as a stone or stone fragment, are provided for convenience and not intended to limit this disclosure. Accordingly, the concepts described herein may be utilized for any analogous medical device or system, e.g., where the medical procedure includes visualization of a target site.

The medical devices, medical systems, and related methods discussed herein may facilitate visualization of a target site under variable circumstances of a medical procedure. Such medical procedures may include, for example, breaking apart (e.g., fragmenting) a material (e.g. stones, etc.) for removal, and manipulating and/or treating fluid or tissue at the target site. During an exemplary procedure, obstructions to the field of view of an imaging device (e.g., camera) may be analyzed to inform adjusting parameters of a medical device or actions taken by the user to increase visualization. Such obstructions may include fluids, such as blood, urine, bile, or other bodily secretions, and/or particulates, such as stone fragments, stone dust, or tissue debris. For example, during an exemplary procedure that includes ablating tissue, such as benign prostatic hyperplasia treatment ablating prostate tissue, tissue debris generated during the procedure may remain within the field of view of the imaging device. Determining and analyzing metrics associated with obstructions may help a medical professional to efficiently remove or reduce material(s) impairing visualization. This in turn may help to reduce the risk of injury to a patient during a procedure, reduce procedure time, and/or increase accuracy of material removal, among other possible benefits.

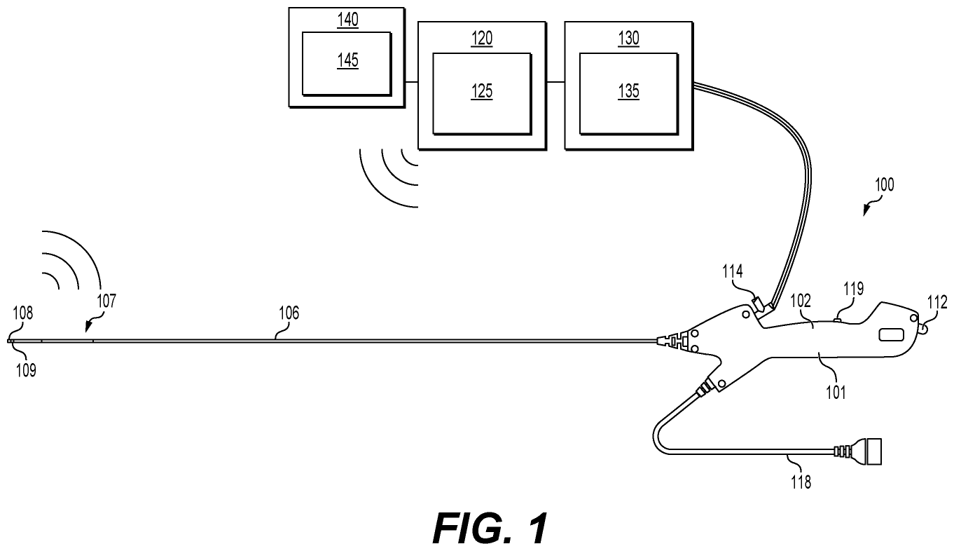

FIG. 1 depicts aspects of an exemplary medical system 100 according to the present disclosure. Medical system 100 includes a medical device 101. Medical device 101 may be a scope such as a ureteroscope or an endoscope. Medical device 101 may include a handle 102 and a shaft 106. Handle 102 may be configured for gripping and use by an operator (e.g., a medical professional). Shaft 106 may extend distally from a proximal end of shaft 106. Shaft 106 includes a distal portion 107, which includes a distal tip 108. Distal tip 108 may include an imaging device 109 and a light source. Imaging device 109 may include, for example, a camera. Light source may include, for example, one or more light emitting diodes (LEDs). Imaging device 109 is in communication with a processor 120 of medical system 100. Processor 120 may be communicatively connected (via wired or wireless connection) to a fluid management system (FMS) 130 of medical system 100. Processor 120 may be configured to monitor and control a fluid flow from FMS 130 based on data from imaging device 109.

At least a portion of shaft 106 may be flexible, for example, to facilitate navigation through a subject's (e.g., patient's) anatomy, e.g., via an articulation section. Shaft 106 may include one or more lumens or channels. For example, shaft 106 may include a working channel configured to receive a medical instrument therethrough. One or more other channels may be connected to or are connectable with a fluid source or vacuum source for delivery of fluid (e.g., irrigation fluid such as saline including isotonic, hypotonic, and hypertonic saline solutions) or application of suction (negative pressure), respectively, at a target site. Other channels may house control wires to navigate shaft 106 or electronic connections to power and/or transfer data from imaging device 109 and/or the light source(s). In some examples, shaft 106 may define a working channel and at least one second channel, e.g., a fluid channel or aspiration channel. The working channel may be capable of receiving a medical instrument (the medical instrument optionally including an end effector) or a laser device to deliver laser energy at a target site proximate the distal end of shaft 106. The second channel may be used for fluid delivery or aspiration. In some examples, shaft 106 may define a working channel and two second channels, e.g., an aspiration channel and a fluid channel.

Imaging device 109 may be configured to capture one or more image frames/videos or a plurality of image frames/videos for a field of view 200 distal to distal tip 108 of shaft 106. For example, imaging device 109 may be configured to continuously or periodically capture image frames or video during an exemplary medical procedure. Imaging device 109 may relay data via a wired or wireless connection for viewing on a display 140.

Field of view 200 of imaging device 109 may be a forward-facing (distal-facing relative to shaft 106) field of view. In other aspects, imaging device 109 may be positioned such that field of view 200 is side-facing relative to shaft 106. Distal tip 108 of shaft 106 may be navigated or positioned within a body of a patient such that field of view 200 includes a target 230 (e.g., a stone) within or at a target site 240 (e.g., a portion of a bodily lumen or cavity).

Handle 102 of medical device 101 may include a mechanism to control articulation of shaft 106. For example, the mechanism may include an actuator such as lever 112 coupled to distal portion 107 via one or more control wires. For example, lever 112 may be coupled to the control wire(s) such that movement of lever 112 controls articulation and steering of shaft 106 including distal tip 108. It will be appreciated that any suitable actuator(s) may be used in addition or in place of lever 112, such as one or more knobs, buttons, sliders, switches, or joysticks. Handle 102 may include one or more locking mechanisms, for example, to lock a position of lever 112 and, thus, lock an orientation of an articulation portion of shaft 106 (e.g., locking an orientation of distal tip 108). In some aspects, an actuator of handle 102 may be operably coupled to imaging device 109 to control imaging device 109 and capture images. For example, handle 102 may include an actuator 119 that initiates imaging device 109 to capture still images and/or video images.

An umbilicus 118 may extend from handle 102 to provide, for example, power, data, and/or fluids to and/or from handle 102. Umbilicus 118 may connect handle 102 to one or more user interfaces, monitors, control units, displays, etc. In some examples, processor 120 and/or FMS 130 may be operably connected to handle 102 via umbilicus 118.

A port 114 of handle 102 may provide access to one or more channels of shaft 106. For example, port 114 may be fluidly connected to the fluid channel of shaft 106. Optionally, handle 102 may include one or more ports in addition to port 114, that may be in fluid communication with one or more other channels of shaft 106, e.g., providing access to the working channel of shaft 106 for extending one or more instruments through the working channel and to a distal opening of the working channel.

As mentioned above, medical system 100 may include processor 120 and FMS 130. Processor 120 may be communicatively connected (e.g., via wired or wireless connections) to FMS 130. FMS 130 may be configured to provide fluid flow (e.g., irrigation fluid) via a pump 135 through the fluid channel of shaft 106 to a distal opening of shaft 106. FMS 130 may include a fluid conduit 150 connected with the fluid channel via port 114. Processor 120 may be configured to control (e.g., maintain or adjust) one or more parameter values of fluid flow from FMS 130 to medical device 101, such as, e.g., volumetric flow rate, pressure, and/or temperature, by providing data (e.g., a signal) to FMS 130. Processor 120 may include a memory 125 configured to store data, e.g., data corresponding to image frames captured by imaging device 109.

Display 140 may be configured to show a user interface 145, e.g., including field of view 200 of imaging device 109 during a medical procedure. User interface 145 may include a current or instantaneous image frame, or live stream video, from imaging device 109, corresponding to field of view 200. User interface 145 may include a parameter indicator 210 superimposed over field of view 200, e.g., to display one or more parameters of interest. According to aspects of the disclosure, user interface 145 may include a plurality of parameter indicators 210, each corresponding to a different parameter of interest. Processor 120 may be configured to update parameter indicator 210 based on image frames captured by imaging device 109.

Parameter indicator 210 may include a status indicator such as a segmented bar or “loading bar” and a numeric output corresponding to the status. It should be understood that parameter indicator 210 may include any suitable graphical representation such as a static or animated graphic. Exemplary parameter indicators 210 include, but are not limited to, throbbers such as progress rings and loading bars, pie charts, color indicators (e.g., stoplight), percentages, fractions, numbers (integers or other magnitude), etc. corresponding to parameter value(s). The parameter value(s) may be an individual parameter value or a plurality of parameter values. In some examples, the parameter indicator 210 may include a graphical representation corresponding to a plurality of parameter values over time.

Processor 120 may be configured to control one or more operating parameters of system 100. For example, processor 120 may be configured to control fluid flow from FMS 130 to handle 102 and shaft 106. Fluid control may be based on data provided by imaging device 109, e.g., analyzed by one or more algorithms of processor 120. For example, fluid control may be based on (e.g., determined and/or adjusted on) one image frame from a point in time during a procedure. The image frame may be a current (instant) image frame or one image frame from a point of interest during the procedure. Additionally or alternatively, fluid control may be based on a plurality of image frames (including, e.g., a video) from a period of time during a procedure.

According to some aspects, FMS 130 may be configured to provide aspiration (e.g., negative pressure, or suction) via a pump 135 through a channel of shaft 106 (e.g., through the fluid channel in a step separate from delivering fluid or a separate channel serving as an aspiration channel) of shaft 106 to a distal opening of shaft 106. For example, processor 120 may be configured to control (e.g., maintain or adjust) one or more parameter values of aspiration (e.g., vacuum pressure) from FMS 130 to medical device 101 by providing data to FMS 130. In some instances, pump 135 may be configured to provide fluid flow in a first setting of step of a procedure and provide aspiration in a second setting or preceding or subsequent step of the procedure. In some aspects, FMS 130 may include a vacuum source fluidly connected to the aspiration channel to provide negative pressure. Processor 120 may be configured to adjust vacuum pressure such that fluid flow out of a distal opening of the shaft (e.g., in a fluid channel) is approximately equal to flow of fluid(s) and/or other materials into a distal opening of the shaft (e.g., in an aspiration channel).

Additionally or alternatively to controlling fluid and/or vacuum pressure, in some examples, processor 120 may be configured to control one or more operating parameters of a laser device or the end effector of a medical instrument within the working channel of shaft 106 based on data provided by imaging device 109. For example, the processor 120 may be configured to monitor and/or control an intensity, frequency, power level, etc., of laser energy provided by the laser device based on data from imaging device 109.

Each image frame captured by imaging device 109 may correspond to a point in time during a medical procedure. Optionally, imaging device 109 and/or processor 120 may provide a time stamp or time-value for each captured image frame.

Processor 120 may include an algorithm (e.g., classification program or module) to analyze and classify image frames, e.g., including quantitative metrics providing an assessment of the clarity of the visualization within the image frames. This quantitative assessment may be used for a user to take actions and/or for electronic connection to one or more secondary devices (e.g., FMS 130, a laser device, a medical instrument, etc.) to take an action to control (e.g., maintain or adjust) properties of the secondary device, such as controlling fluid (using FMS 130) through medical device 101. For example, the quantitative assessment may guide a user to manually adjust fluid flow (optionally using FMS 130) or may automatically adjust fluid flow using FMS 130. Moreover, the use of quantitative metrics may provide for more precise control of FMS 130, a faster response in adjusting parameters to improve visualization, and greater clarity in field of view 200 as compared to a relative determination. Further, in some examples, the use of quantitative metrics to accord a visualization score may provide for automated control of fluid flow within predetermined parameters in order to increase visibility, such that a medial professional need not manually adjust fluid to provide greater clarity before continuing a medical procedure.

For example, processor 120 may receive a plurality of image frames in real-time or saved to memory 125 and analyze each image frame, a plurality of image frames, or a plurality of image frames from a period of time during the procedure in order to accord each image frame and/or the plurality of image frames a visualization score. Based on the visualization score(s), processor 120 may automatically cause FMS 130 to adjust parameters of fluid flow. Additionally, or alternatively, processor 120 may provide visualization data to display 140. A user (medical professional) may manually adjust parameters of fluid flow based on the displayed data using FMS 130. Optionally, manual input from the user may override any automatic adjustments to FMS 130 from processor 120.

The visualization scores of the image frames may provide an indication of the degree to which a user is able to accurately view target site 240. Image frames wherein field of view 200 is obstructed due to the presence of various materials (blood, other bodily fluids, tissues, stone fragments, etc.) on imaging device 109 or sufficiently close to imaging device 109 so as to hinder viewing of target site 240. The visualization score of a given image frame may take into account various features of the image frame, e.g., color differences among pixels across the image frame, the presence or absence of sharp edges, variations between consecutive image frames, etc.

The visualization score (which may be considered inversely as a turbidity level) for an image frame may be determined by the algorithm analyzing the image frame to detect the presence and concentration of material 250 within the field of view 200. As mentioned above, material 250 may include dust (e.g., from stone fragmentation) or other particulates, blood, and/or bodily secretions, that may obscure field of view 200. Processor 120 may generate a low visualization score (e.g., less than 50%, e.g., between 0 and 49.999%) when turbidity-levels are high, e.g., reflecting a blurry or otherwise obscured image due to the presence of fluids and/or particulate matter on or close to imaging device 109. Processor 120 may generate a high visualization score (e.g., greater than 50%, e.g., between 50% and 100%) when turbidity-levels are low.

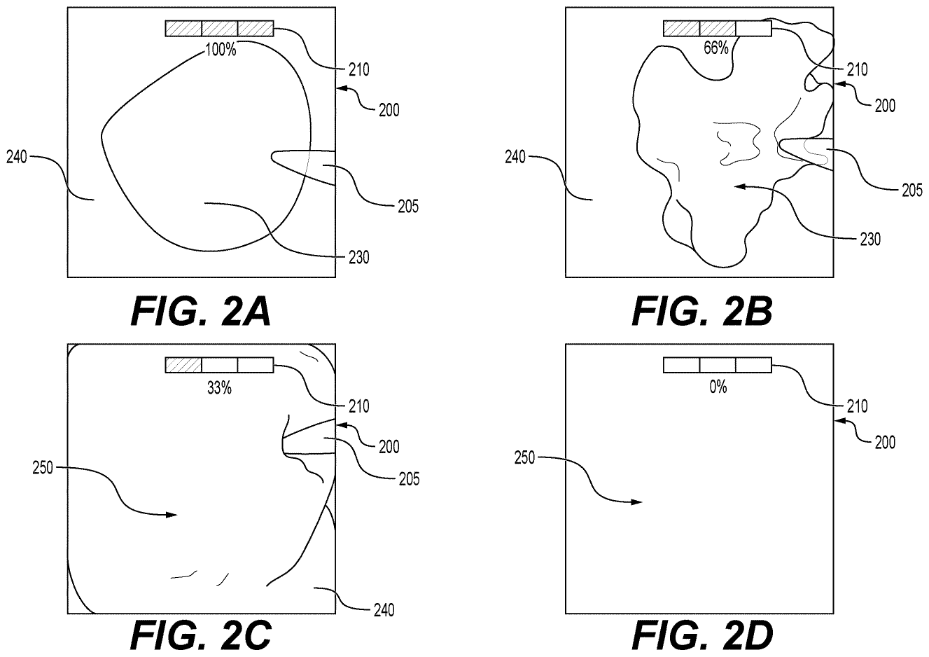

FIGS. 2A-2D illustrate exemplary fields of view 200 with varying clarity of features of a target site, demonstrating different visualization scores generated (e.g., calculated) by processor 120. The field of view 200 may include a distal portion 205 of a medical device. The distal portion may correspond to a medical instrument extended through a channel (e.g., working channel) of shaft 106. For example, field of view 200 depicted in FIG. 2A is the clearest (e.g., lowest turbidity), corresponding to the highest visualization score of 100%, as indicated by parameter indicator 210. Parameter indicator 210 in FIG. 2A includes a throbber (e.g., loading bar) with three of three segments highlighted indicating a visualization score of approximately 100%. Conversely, field of view 200 depicted in FIG. 2D is the most obscured (e.g., highest turbidity) corresponding to the lowest visualization score of 0%. Accordingly, in FIG. 2D, the throbber of parameter indicator 210 shows zero of three segments highlighted indicating a visualization score of approximately 0%. FIGS. 2B and 2C illustrate examples of interim visualization scores of 66% and 33%, respectively. The throbber of parameter indicator 210 shows two of three segments highlighted in FIG. 2B indicating a visualization score of approximately 66%, and shows one of three segments highlighted in FIG. 2C indicating a visualization score of approximately 33%. These visualization scores reflect application of the algorithm of processor 120, e.g., analyzing pixels within image frames.

According to aspects of this disclosure, processor 120 may be configured to generate (e.g., calculate) an aggregate visualization score (also referred to herein as an aggregate score) for a plurality of image frames or video, e.g., as an average, median, or range of individual visualization scores of individual image frames. That is, an aggregate score for a plurality of image frames or video may correspond to the average, median, or a range of individual visualization scores for the plurality of image frames or video. For example, processor 120 may generate an aggregate score for a plurality of image frames from a period of interest (e.g., a time interval) during a procedure. Additionally or alternatively, processor 120 may generate an aggregate score for a plurality of image frames from the beginning of a procedure to a desired point in time during the procedure. Aggregate scores (e.g., a moving mean or median) of a plurality of image frames or video may be evaluated on the same scale as individual visualization scores. For example, where the aggregate score is a moving average of visualization scores from a plurality of image frames, an aggregate score calculated by processor 120 may be low (e.g., less than 50%, e.g., between 0 and 49.999%) when turbidity-levels are high and may be high (e.g., greater than 50%, e.g., between 50% and 100%) when turbidity-levels are low. In some examples, processor 120 may calculate visualization scores for a plurality of image frames or video from a period of time during a procedure, and then calculate an aggregate score for the same period of time.

Processor 120 may be configured to provide real-time signals to FMS 130 to adjust one or more parameters of fluid flow delivered by FMS 130 based on visualization score data including individual visualization scores and/or aggregate scores. According to aspects of this disclosure, processor 120 may be configured to compare visualization score data to a predetermined threshold, e.g., saved in memory 125. In some aspects, processor 120 may be configured to compare a derived parameter value of the visualization score data to a predetermined threshold.

In an example, processor 120 may compare a visualization score for a current (instant) image frame, or an aggregate score of a plurality of image frames to a threshold (e.g., a predetermined threshold). The threshold saved by memory 125 may be numerical value or a numerical range. In some examples, the threshold may correspond to a visualization score at which a medical professional would take an action such as pause a procedure due to poor visualization of target 230 and/or other features of target site 240. Additionally, or alternatively, the threshold may be at least partially based on specifications of components of medical device 101 and/or system 100. The threshold may be determined based on known properties of medical device 101, fluid (e.g., irrigation fluid provided by delivery channel of shaft 106), and/or properties of FMS 130. In some examples, a different or same predetermined threshold may be used in the respective comparisons of an individual visualization score and an aggregate score.

After comparing the visualization score for a given image frame or an aggregate score for a given plurality of image frames or video to the threshold, processor 120 may determine whether and how to modify parameter values of FMS 130. For example, if the visualization score or aggregate score of the image frame(s) or video is less than or equal to the predetermined threshold stored by memory 125, processor 120 may instruct FMS 130 to adjust one or more parameter values of fluid flow and/or vacuum pressure. In some examples, processor 120 may be configured to instruct FMS 130 to increase the volumetric flow rate of fluid to target site 240, e.g., to flush out or otherwise remove material 250 from field of view 200. Where the threshold is a continuous or discrete numerical range, processor 120 may be configured to maintain the current parameters of FMS 130 controlling fluid flow if the visualization score is within the range of upper and lower bounds of the threshold. Further, if the visualization score is greater than an upper bound of the threshold, processor 120 may be configured to instruct FMS 130 decrease the volumetric flow rate of fluid. If the visualization score is lower than the lower bound of the threshold, processor 120 may be configured to instruct FMS 130 to increase the volumetric flow rate of fluid from FMS 130.

According to some aspects of this disclosure, using visualization data and time data of one or more image frames captured thus far during a procedure, processor 120 may be configured to determine an amount of elapsed time that a visualization score of an instant image frame is below the threshold stored by memory 125. During or after the procedure, processor 120 may be configured to display the amount of elapsed time below the threshold on display 140. The amount of time below the threshold may correspond to the amount of time that the user (medical professional) paused the procedure or otherwise took action due to low visualization. Conversely, an amount of time above the threshold may correspond to the amount of time that the user is actively performing the procedure, e.g., workable time. Processor 120 may be configured to instruct FMS 130 to adjust one or more parameters of fluid flow based on the amount of elapsed time below the threshold. For example, processor 120 may be configured to instruct FMS 130 to increase volumetric flow rate if the amount of elapsed time below the threshold exceeds a predetermined timing threshold.

According to aspects of the disclosure, processor 120 may be configured to derive a clarity value based on the visualization score or aggregate score (e.g., a moving average of visualization scores). For example, processor 120 may be configured to convert a visualization score or aggregate score to an integer value, x, e.g., on scale from 1 to 4 (e.g., a clarity of 1, 2, 3, or 4). An image frame having a clarity with an integer value of 4 may indicate a high visualization score, whereas an image frame having clarity with an integer value of 1 may indicate a low visualization score. The clarity value may reflect a moving average. For example, a plurality of image frames having a clarity with an integer value of 4 may indicate a high moving average of visualization scores, whereas having a clarity with an integer value of 1 may indicate a low moving average of visualization scores. The aggregate score may include a moving average of visualization scores of images from a period of time during the procedure. For example, the aggregate score may be a moving average of the visualization scores of image frames captured within a given time period (e.g., three seconds) preceding a current point in time. Using the clarity integer value, x, processor 120 may be configured to calculate a percent clarity, % c, of an image frame using the equation below.

% c=(x−1)/3

Percent clarity % c may be graphically displayed by parameter indicator 210 of user interface 145. For example, field of view 200 depicted in FIG. 2A may correspond to a point in time before laser energy is delivered to target 230 (e.g., to ablate or fragment a kidney stone). FIGS. 2B-2D may correspond to points in time after target 230 has been ablated or otherwise fragmented such that particulate matter is released. FIG. 2A depicts field of view 200 without or largely absent of interfering material.

Processor 120 may be configured to calculate a standard deviation (e.g., as an aggregate score) of the visualization scores for a plurality of image frames. The standard deviation may provide a measure of how consistent the visualization quality has been over time. Instead of or in addition to calculating the standard deviation of the visualization scores, processor 120 may be configured to calculate a coefficient of variance. Processor 120 may be configured to compare the calculated standard deviation to a predetermined threshold of the standard deviation or coefficient of variance, respectively. This threshold may represent an acceptable level of variability in visualization quality of a plurality of images over time during a procedure. The threshold may be determined or selected based visualization scores from a plurality of images from one or more of previously completed procedure(s) performed by the same or a different user. If the calculated standard deviation is below the threshold, it may indicate that visualization has been relatively consistent. Conversely, if the standard deviation exceeds the threshold, it may suggest significant fluctuations in visualization quality. Further, based on this comparison, processor 120 may instruct FMS 130 to adjust fluid flow. For example, if the standard deviation is above the predetermined threshold, FMS 130 may increase the fluid flow rate to stabilize visualization quality. In some cases, if the standard deviation is significantly below the threshold, processor 120 may instruct FMS 130 to decrease the fluid flow rate, e.g., to conserve resources while maintaining adequate visualization. In examples using the coefficient of variance, processor 120 may be configured to calculate a coefficient of variance, compare the calculated coefficient of variance to a predetermined threshold, and instruct FMS 130 to adjust or maintain parameters of fluid flow.

According to aspects of the disclosure, processor 120 may be configured to instruct FMS 130 to adjust one or more parameters values of fluid based on a rate at which visualization scores of one or more image frames captured thus far during the procedure increase or decrease over time. Memory 125 of processor 120 may include a predetermined threshold for a declining rate of visualization scores and another predetermined threshold for an increasing rate of visualization scores. In at least one example, processor 120 may be configured to compare an increasing rate of visualization scores to the respective predetermined threshold. If the increasing rate is less than the predetermined threshold, processor 120 may be configured to instruct FMS 130 to maintain the fluid flow parameters. If the increasing rate is greater than the predetermined threshold, processor 120 may be configured to instruct FMS 130 to decrease volumetric flow rate. An increasing rate of visualization may correspond to faster flushing of material 250 from target site 240. Further, if the rate of visualization is greater than an upper bound of the predetermined threshold, processor 120 may be configured to instruct FMS 130 to decrease the volumetric flow rate of fluid. If the rate of visualization is lower than a lower bound of the threshold, processor 120 may be configured to instruct FMS 130 to increase the volumetric flow rate of fluid from FMS 130. In instances where the rate of visualization is lower than the lower bound of the threshold, processor 120 may be configured to increase volumetric flow rate to a flow rate configured to cause visualization scores of future image frames to increase to a value within a range of the threshold but not exceed an upper bound of the range.

According to some aspects of this disclosure, using visualization data and time data of one or more image frames captured during a procedure, processor 120 may be configured to calculate an area under the curve. The area under the curve (e.g., visualization score over time) may correspond to a visualization dosage metric or score. The visualization dosage score may be used to inform a user of the differences in aggregate scores for different periods of time during a procedure. For example, the visualization dosage score may be shown on display 140 or otherwise communicated (e.g., via an alert) to a user. In some examples, one or more previous visualization dosage scores from a previously completed procedure or an earlier time interval from a current procedure may inform actions taken by processor 120 or the user to adjust fluid flow rate to change visualization to be more consistent across a larger period of time. In at least one example, processor 120 may adjust flow rate in real time based on a comparison between a previous visualization dosage from a controlled setting with known variables or a completed procedure, and a visualization dosage from a current procedure. In at least one example, a visualization dosage from a completed procedure may be compared to a previous visualization dosage from a controlled setting or a different, completed procedure, and based on this comparison, processor 120 may be configured to adjust fluid flow rate in future procedures.

Optionally, processor 120 may be configured to issue an alert upon one or more of the abovementioned determinations (e.g., visualization score, rate of visualization, visualization dosage, standard deviation, time elapsed below a threshold, etc.) falling below or exceeding a predetermined threshold stored by memory 125. The alert may be visual, auditory, or haptic, and may be shown on display 140 or transmitted to another device. For example, if the percent clarity of an instant image frame falls below the predetermined threshold, processor 120 may trigger a visual alert on display 140, such as a flashing icon or a change in color of the parameter indicator 210. In some cases, the alert may include a recommendation for the user (medical professional), such as suggesting an increase in fluid flow rate or a pause in the procedure to allow for improved visualization.

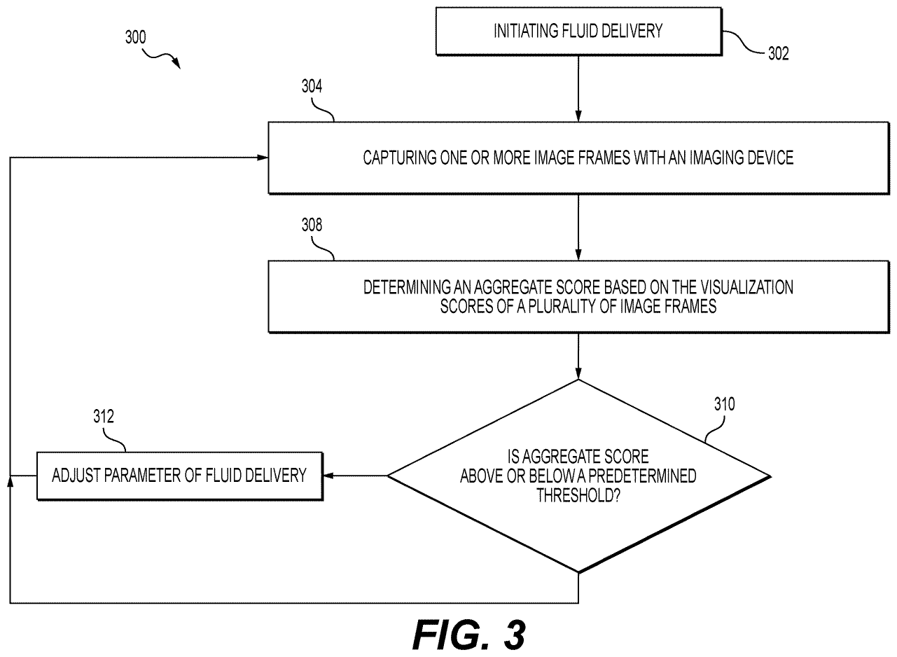

FIG. 3 depicts a flow chart of an exemplary method 300 for monitoring and adjusting fluid delivery during a medical procedure using medical system 100. Method 300 includes a step 302 of initiating fluid delivery. Step 304 may include capturing one or more image frames with imaging device 109. Step 304 may include processor 120 generating visualization scores with an algorithm (e.g., an image-classification program) based on the one or more image frames as discussed above. For example, processor 120 may generate a visualization score for each image frame captured by imaging device 109. During the procedure, a visualization score may be generated for each image frame received from imaging device 109.

Step 308 includes determining (generating, calculating) an aggregate score (e.g., mean, median, mode, standard deviation, coefficient of variance, etc.) based on the visualization scores of a plurality of image frames. In some examples, instead of an aggregate score being determined based on the visualization scores, one or more of the above visualization metrics (e.g., time elapsed, visualization dosage, rate of change, etc.) may be determined.

Method 300 may include a decision node or decision point 310 of determining if the aggregate score is above or below a predetermined threshold. Processor 120 may be configured to compare the aggregate score to the corresponding predetermined threshold. If the aggregate score is above the predetermined threshold (following the “Yes” branch from decision step 310), this indicates that visualization of field of view 200 is sufficient, and method 300 may return to step 304 to continue capturing images without adjusting parameters of FMS 130. If the aggregate score is below the predetermined threshold (following the “No” branch from decision step 310), this indicates that visualization of field of view 200 is insufficient, and the method 300 proceeds to step 312 to adjust fluid delivery parameters of FMS 130, such as increasing volumetric flow rate. In aspects using other disclosed visualization metrics (e.g., time elapsed, visualization dosage, rate of change, etc.), the selected visualization metric may be compared to a corresponding predetermined threshold.

According to aspects of the disclosure, while processor 120 may generally be described as performing functions or tasks during a medical procedure, it should be understood that processor 120 may also be configured to receive data (e.g., raw data) including visualization data and time data at the end of a procedure or from a previous procedure and calculate one or more of the metrics described above based on the data. These calculated metrics may be shown on display 140 after the procedure concludes. In some cases, the user may compare the metrics to past procedures or from different medical systems or devices. This post-procedure analysis capability may allow for retrospective evaluation of procedure performance, identification of trends within different periods of time within a procedure or across multiple procedures, and/or comparison of different medical systems or devices. Quantitative visualization metrics from different procedural cases may allow for comparison and evaluation of performance in terms of visualization according to other variables. These variables may include, e.g., different operating environments, procedural differences, different patient populations, and/or different users (treating physicians). The visualization metrics post procedure may be used in combination with quantification of other actions during the same procedure, such as pausing to improve visualization, e.g., to draw correlations between visualization and other actions or metrics. Quantitative visualization metrics from procedures using different medical devices may be useful to evaluate medical device changes impacting performance to quantitatively measure performance improvements, including between prototype iterations of a medical device.

While principles of this disclosure are described herein with reference to illustrative examples for particular applications, it should be understood that the disclosure is not limited thereto. Those having ordinary skill in the art and access to the teachings provided herein will recognize additional modifications, applications, and substitution of equivalents all fall within the scope of the examples described herein. Additionally, a variety of elements from each of these embodiments can be combined to achieve a same or similar result as one or more of the disclosed embodiments. Accordingly, the invention is not to be considered as limited by the foregoing description.

What is claimed is:

1. A medical system comprising:
a handle including a port configured to be coupled to a fluid management system;
a shaft extending distally from the handle to a distal end of the shaft, wherein the shaft defines a fluid channel in communication with the port, wherein a distal portion of the shaft includes an imaging device; and
a processor communicatively connected to the imaging device, wherein the processor is configured to:
receive one or more image frames from the imaging device,
analyze pixels for each frame of the one or more image frames,
calculate a visualization score for the one or more image frames based on the analyzed pixels of the respective image, and
relay instructions to the fluid management system to adjust a fluid flow through the fluid channel based on the visualization score.

2. The medical system of claim 1, wherein the one or more image frames includes a plurality of image frames and the processor is configured to calculate an aggregate score of a plurality of visualization scores of the plurality of image frames.

3. The medical system of claim 2, wherein the aggregate score is a mean, median, range, or standard deviation of the plurality of visualization scores.

4. The medical system of claim 1, wherein the processor is configured to compare the visualization score to a predetermined threshold.

5. The medical system of claim 4, wherein the processor is configured to instruct the fluid management system to increase fluid flow through the fluid channel if the visualization score is less than or equal to the predetermined threshold.

6. The medical system of claim 4, wherein the processor is configured to instruct the fluid management system to maintain or decrease fluid flow through the fluid channel if the visualization score is greater than the predetermined threshold.

7. The medical system of claim 4, wherein the processor is configured to generate an alert if the visualization score is less than or equal to the predetermined threshold.

8. The medical system of claim 4, wherein the predetermined threshold is a standard deviation of one or more visualization scores from a previous medical procedure.

9. The medical system of claim 1, wherein the processor is configured to calculate an amount of time elapsed during which the visualization score is less than a predetermined threshold.

10. The medical system of claim 9, wherein the processor is configured to instruct the fluid management system to increase fluid flow through the fluid channel when the time elapsed exceeds the predetermined threshold.

11. The medical system of claim 1, further comprising a display in communication with the processor.

12. The medical system of claim 11, wherein the display includes a user interface.

13. The medical system of claim 1, wherein the shaft includes a working channel that receives a laser device, and wherein the processor is configured to relay instructions to the laser device to adjust laser energy delivered at the distal end of the shaft based on the visualization score.

14. The medical system of claim 1, wherein the processor is configured to relay instructions to the fluid management system to adjust a pressure of the fluid flow through the fluid channel.

15. A medical system comprising:
a handle including a port configured to be coupled to a fluid management system;
a shaft extending distally from the handle to a distal end of the shaft, wherein the shaft defines a fluid channel in communication with the port, wherein a distal portion of the shaft includes an imaging device;
a display; and
a processor communicatively connected to the imaging device and the display, wherein the processor is configured to:
receive a plurality of image frames from the imaging device,
analyze pixels for each frame of the plurality of image frames,
calculate a visualization score for each image frame based on the analyzed pixels of the respective image, and
relay instructions to the fluid management system to adjust a fluid flow through the fluid channel based on the visualization scores.

16. The medical system of claim 15, wherein the processor includes a memory that stores a threshold visualization score.

17. The medical system of claim 15, wherein the processor is configured to calculate an amount of time elapsed during which the visualization score is less than a predetermined threshold.

18. The medical system of claim 17, wherein the processor is configured to instruct the fluid management system to increase fluid flow through the fluid channel when the time elapsed exceeds the predetermined threshold.

19. The medical system of claim 15, wherein the display includes a user interface, wherein the user interface includes a parameter indicator associated with a parameter of the fluid flow or a visualization score of at least one image frame of the plurality of image frames.

20. A medical system comprising:
a handle including a port configured to be coupled to a fluid management system;
a shaft extending distally from the handle to a distal end of the shaft, wherein the shaft defines a fluid channel in communication with the port, wherein a distal portion of the shaft includes an imaging device;
a display; and
a processor including a memory, the processor being communicatively connected to the imaging device and the display, wherein the processor is configured to:
receive a plurality of image frames from the imaging device,
analyze pixels for each frame of the plurality of image frames,
calculate a visualization score for each image frame based on the analyzed pixels of the respective image,
compare the visualization score to a predetermined threshold stored by the memory, and
relay instructions to the fluid management system to adjust a fluid flow through the fluid channel based on the visualization scores.
